(19) United States
(12) Patent Application Publication
(10) Pub. No.: US 2009/0197085 A1
(43) Pub. Date: Aug. 6, 2009

(54) ORGANIC NANOPARTICLES AND METHOD OF PREPARATION THEREOF

(76) Inventor: Nicholas V. Coppa, Malvern, PA (US)

Correspondence Address: Nicholas V Coppa, 15 North Bacton Hill Road, Malvern, PA, 19355, US

(21) Appl. No.: 12/309,890

(22) PCT Filed: Aug. 6, 2007
(86) PCT No.: PCT/US2007/017496

§ 371 Date: Feb. 2, 2009

Related U.S. Application Data
(60) Provisional application No. 60/836,067, filed on Aug. 7, 2006.

(51) Int. Cl. B32B 5/02; C07D 307/62

(52) U.S. Cl. 428/402; 549/315; 977/775

(57) ABSTRACT

The invention relates to organic nanopowders and methods for their production, including ascorbic acid nanoparticles and nanopowder salts of ascorbic acid, such as, calcium ascorbate nanopowders. Such organic nanopowders may have utility in cosmetics, pharmaceutical preparations and nutrition. The invention additionally relates to methods for producing ascorbic acid nanoparticles and calcium ascorbate nanopowders. The method of preparation of ascorbic acid or calcium ascorbate nanopowders involves: (i) preparing an solution including an organic compound solute and a solvent to disperse or dissolve the organic compound, and (ii) removal or separation of the solvent in such a manner so as to limit the growth of the organic solute particles to nanometer range which is typically below 500 nm but preferably 100 nm or less.

### CROSS-REFERENCE TO RELATED APPLICATION

This application claims priority to U.S. Provisional patent application Ser. No. 60/836,067, filed on Aug. 7, 2006, the complete disclosure of which is herein incorporated by reference.

### BACKGROUND OF THE INVENTION

1. Field of the Invention

The invention relates to organic nanoparticles and including salts thereof, such as calcium ascorbate nanopowders, and methods for their production.

2. Brief Description of the Related Art.

Ascorbic acid is a water soluble organic acid, and exists in enantiomeric forms, the L-enantiomer of which is generally referred to a Vitamin C. The chemical name for ascorbic acid is: 2-oxo-L-threo-hexono-1,4-lactone-2,3-enediol or (R)-3,4-dihydroxy-5-((S)-1,2-dihydroxyethyl)furan-2(5H)-one. Ascorbic acid is useful as an antioxidant. Among the uses of ascorbic acid, and its sodium, calcium and potassium salts, are as anti-oxidants for a food additive. Ascorbic acid is also used in cosmetic formulations, including as a pH adjuster.

### SUMMARY OF THE INVENTION

Organic nanoparticles and salts thereof, such as sodium, potassium and calcium ascorbate nanopowders, and methods for their production are provided. The organic nanoparticles may be used in a variety of industrial and consumer products, including, for example, in cosmetics, pharmaceutical preparations, nutrition, such as nutritional additives, components and/or supplements. The methods and nanoparticle products produced may facilitate the rapid solid state synthesis of materials, according to some embodiments, without the need for the use of solvents.

Organic nanoparticles and salts of organic are produced having particle sizes less than 100 nanometers, and may be prepared with particles sizes as small as less than 10 nanometers.

### DETAILED DESCRIPTION OF THE INVENTION

Methods for the preparation of organic nanopowders are provided in conjunction with the production of an exemplary organic nanopowder, such as ascorbic acid or calcium ascorbate nanopowders. According to preferred embodiments, organic nanoparticles may be produced by: (i) preparing an solution including an organic compound solute and a solvent to disperse or dissolve the organic compound, and (ii) removal or separation of the solvent in such a manner so as to limit the growth of the organic solute particles to nanometer range which is typically below 500 nm but preferably 100 nm or less. These two processes may be effected in many ways including by freeze drying, flash evaporation, vacuum flash evaporation and other methods.

One embodiment of the present invention involves freeze drying. In that method a solution containing an organic compound is made. For example, solutions containing organic acids which are water soluble may be made by dissolving the acid in deionized water. According to embodiments of the invention, the method may also include degassing the solution to remove dissolved gasses that might be present. The solution containing the organic compounds, such as, for example, organic acids, are frozen. Preferably, droplets of the solution are frozen. One preferred freezing method involves atomizing the solution and subjecting the atomized solution to a temperature sufficient to freeze the solution droplets. In preferred embodiments, the atomized solution may be dispersed into the presence of an inert freezing medium, such as, for example, liquid nitrogen, or more particularly, stirred liquid nitrogen. It is preferred that the inert freezing medium be maintained at a temperature relative to the solution in order to facilitate the independent freezing of solvent droplets.

The method and apparatus may include a facilitating means, such as heating device, such as a heater, heating element, coil, or other suitable element, to facilitate the movement of the solvent from a dispenser, such as a nozzle, from which the solvent is dispensed. According to preferred embodiments of the invention, the solvent may be delivered to a freezing chamber, such as, for example, a glass tube. The freezing chamber preferably is constructed from an inert composition relative to the solvent and freezing medium. The solvent may be delivered through a moveable delivery mechanism which permits the positioning of the nozzle for delivery of the solvent at a desired position. A positionable nozzle may be used to deliver the solvent to a chamber for freezing.

An example showing a preferred method for producing ascorbic acid nanoparticles is set forth herein. Though the descriptions and examples provided discuss preferred embodiments where ascorbic acid nanoparticles are produced, the preparation of other organic nanopowders may be carried out in accordance with the methods described herein.

Preparation of Ascorbic Acid Solutions: Solutions were prepared using deionized water with a resistivity greater than 18 MΩ cm⁻¹. Ascorbic acid was solubilized in the water. An amount of ascorbic acid was used to make up the concentration of the solutions to 0.18 M. All solutions were degassed by passing the entire solution through a glass fritted funnel attached to a vacuum sidearm flask attached to a trapped water aspirator.

Preparation of Calcium Ascorbate Solutions: Solutions were prepared using distilled deionized water with a resistivity greater than 18 MΩ cm⁻¹ similar to the ascorbic acid solutions described herein, using, however, calcium ascorbate. The concentration of the solutions was 0.18 M. All solutions were degassed by passing the entire solution through a glass fritted funnel attached to a vacuum sidearm flask attached to a trapped water aspirator.

Freezing of Solution Aerosol: The prepared solutions were subjected to a freezing step. The solution was frozen by atomizing it using an ultrasonic spray nozzle (Sono Tek, 8700-120MS/PS-8S) and allowing the aerosol to fall into stirred liquid nitrogen. The nozzle was operated at 4.8 to 5.0 Watts and a flow rate of about 11 h⁻¹. The particle size of the spray under these conditions as specified by the manufacturer was on the order of 10 ml. Each droplet of the spray is assumed to freeze independently of the others and in this way freezing is thought to be instantaneous. Calculations on model droplets using methods found in A. V. Luikov, “Analytical Heat Diffusion Theory,” (Academic Press, New York, 1968) suggest the freezing rate was on the order of 10⁶° C. s⁻¹.

The spray nozzle was fitted with a small flexible Teflon coated heater controlled by a temperature controller (Omega, 4001 KC) and maintained at 65 C. for the purposes of maintaining the temperature of the solution exiting the tip of the nozzle at 25 C. while in close proximity to the liquid nitrogen surface (−195 C.). Without this heater the orifice of the spray nozzle would cool causing the solution within to freeze. The temperature of the solution exiting from the tip of the nozzle situated above the surface of the liquid nitrogen was measured by using a thermocouple and digital thermometer, (Omega). The use of lower temperatures was not possible because cooling the solution prior to freezing would lead to premature precipitation of the solute. The spray nozzle assembly was suspended from a frame supported system which allowed vertical and horizontal motions for precise control of the position of the assembly over the surface of the liquid nitrogen. The tip of the spray nozzle was maintained 2.5 to 3.0 cm above the surface of the liquid nitrogen by an automatic nitrogen leveling system described below.

The frozen solution was collected in a glass tube (Pyrex, 10 cm×100 cm) fitted with a copper screen (300 mesh) wired to the bottom. At the top of the tube were two clamps which served as supports during the freezing process and as handles for emptying its contents. The tube was suspended into a large 15 L Dewar flask having a viewing sight running its length on opposite sides. The level of liquid nitrogen was automatically maintained at 15+/−0.25 cm from the brim of the Dewar flask using a very precise level controller connected to a 160 L low pressure liquid nitrogen storage tank. Liquid nitrogen entered the Dewar outside the collection tube. In this way the surface of the liquid nitrogen just below the spray nozzle was isolated from the violent sparging caused by intermittent filling of the Dewar. The blades of a mechanical stirrer were positioned 3 cm below the surface of the liquid nitrogen to rapidly mix the aerosol with the nitrogen and prohibit the aerosol from riding on the nitrogen surface.

The solution was fed by gravity into the spray nozzle from a 15 L polyethylene storage bottle with a spigot at its bottom (Nalgene) which was suspended approximately one meter above the nozzle. In this way the change in height of the solution relative to the nozzle orifice as the bottle emptied was small compared to the overall height and therefore the feed pressure of the solution remained relatively constant. A Teflon tube fitted with a stopcock made of the same material connected the storage bottle to the orifice. A liquid sensor consisting of two fine platinum wires was placed in this line which would turn off the power to the nozzle when the tank emptied. This allowed the system to operate unattended while avoiding possible damage to the spray head in the event it became dry.

After the Dewar was full or when the specified amount of solution was frozen, the inner tube containing the frozen solution was removed and its contents poured into shallow precooled teflon coated stainless steel trays. Then these trays were either placed in storage at −32 C. or immediately placed in a sublimation device.

Alternate methods of freezing the solution may be utilized, such as, for example, impinging an aerosol or continuous stream on a cold rotating cylinder or disk, and rapid adiabatic expansion of an aerosol into a vacuum. These methods were applied and the solidified solution remained free of phase separation. Preferred solidification rates associated with solvents were suitable on the order of from about 10² and 10⁶° C. s-1. The lower solidification rate may be employed when the viscosity of the solvent was strongly dependent on the inverse of the absolute temperature, and the higher solidification rates may be utilized where the viscosity of the solution depended little on the absolute temperature.

Sublimation of Frozen Solvent: The aqueous solvent of the frozen ascorbic acid or calcium ascorbate solutions as prepared above was sublimed as follows. A modified commercial laboratory freeze dryer (FTS Systems, FD6-54A-O/TD-2A) operating at reduced pressures was used to complete the drying process and for drying smaller volumes. Typically, pre-drying was unnecessary for the majority of samples prepared in this work. The apparatus included a refrigerated chamber with thermostated Teflon coated stainless steel shelves (−40\T\40 C.) connected by a large orifice to a condenser coil (−55 C.). Each tray had a thermocouple mounted in the center so the temperature of the sample could be remotely monitored. Temperature control of the tray was achieved by the circulation of a heat transfer fluid between the tray holder and refrigeration/heating coils located outside the chamber. The temperature of this fluid was controlled and monitored on the front panel of the system. The sublimator and condenser spaces were continuously kept at a pressure of<30 lm Hg by a high speed rotary oil vacuum pump.

A sample may be subjected to sublimation conditions for a sufficient time so that all or a substantial amount of the solidified solvent may sublime. According to preferred embodiments, typically a sample was kept under the sublimation conditions for a time period proportional to its weight and packing density followed by a slow warming period to room temperature. Samples were considered dry when two criteria were met: (i) The temperature of the tray and the circulating fluid were equal. (ii) Upon raising the temperature of the circulating fluid 2 C., the pressure within the chamber remained constant. When the drying was completed, the chamber was back-filled with dry nitrogen. The ascorbic acid or calcium ascorbate nanopowder product, was removed from the sublimator and quickly transferred, for example, by pouring into a large-mouthed glass vessel and immediately sealed. Transfer of the powder from this temporary container to glass storage bottles was done in a glove box under dry nitrogen at a relative humidity of<20%.

An alternate method was to connect the sublimation device directly to a dry box so that manipulation of the nanopowder product was done in a controlled environment. A further alternate method was to use a vacuum driven device that would remove the nanopowder product directly from the trays. The vacuum driven device was constructed from a long tube, attached to a collection vessel. Within the collection vessel was a means for separating the solid particles from the conveying medium, a tube exiting the means for separating the solid particles from the conveying medium which was connected to a vacuum (low pressure) source. All tubing was made of electrically conducting materials and grounded to the earth. The means for separating the solid particles from the conveying medium involved a porous membrane having porosity sufficiently small to entrap the smallest particles. Other handling methods exist including sublimation in individual vials or containers, automatic stoppering of such containers and the multitude of variations currently used in the food, materials, and pharmaceutical industries for the preparation of sensitive materials. The resulting nanopowder product has a large potential energy driving the reduction of its surface area. According to preferred embodiments of the invention, the nanoparticle products may be handled in a controlled environment. Atmospheric constituents such as moisture may greatly affect the kinetic barriers to the reduction in surface area and the concomitant growth in particle size. The examples of material handling during and after sublimation described herein were not meant to represent an exhaustive of the manner in which the product may be handled during and after sublimation.

The resultant products produced ascorbic acid nanoparticles having particle sizes less than 500 nanometers, and as little as 10 or less nanometers. Product was obtained and analyzed for ascorbic acid nanoparticles of about or several reactions. Ascorbic acid was produced using the methods described herein to obtain ascorbic acid nanoparticles, including ascorbic acid nanoparticles with particle sizes of about less than 500 nanometers, and including ascorbic acid nanoparticles having particle sizes of less than 10 nanometers. The ascorbic acid salts, such as, for example, calcium ascorbate, may be produced having similar particle sizes, that is, less than 500 nanometers, and even less than 10 nanometers.

While the invention has been described with reference to specific embodiments, the description is illustrative and is not to be construed as limiting the scope of the invention. Various modifications and changes may occur to those skilled in the art without departing from the spirit and scope of the invention described herein and as defined by the appended claims.

1. A method for producing organic nanopowder compounds comprising of the following steps:
(a) providing a solution comprising organic solute and a solvent selected from the group consisting of a compatible liquid;
(b) separating the solvent component from the solution while limiting the growth of organic solute particles to provide an organic nanopowder product.

2. The method of claim 1, wherein the step of separating the solvent component includes freezing the solution to effectuate the formation of a solidified material comprising solidified solvent and immobilized organic solute followed by sublimation of solvent.

3. The method of claim 1, wherein the step of separating the solvent component includes evaporation of the solvent component.

4. The method of claim 2, wherein the solution is rapidly solidified so that little or no phase separation occurs.

5. The method of claim 2, wherein during the step of separating the solvent, the solidified material is held at a temperature equal to or less than the eutectic point of the solvent and organic solute binary system.

6. The method of claim 1, wherein the separated particles are collected.

7. The method of claim 1, wherein the solvent is water.

8. The method of claim 1, wherein freezing the organic solution includes aerosolizing the solution.

9. The method of claim 1, wherein freezing is effected by rapid freezing.

10. The method of claim 1, wherein the solution is dispersed into aerosol droplets and rapidly mixed with a cryogenic liquid whereby said cryogenic liquid causes the aerosol droplets to freeze.

11. The method of claim 1, wherein removing the solvent method is accomplished by sublimation of the solvent.

12. The method of claim 1, wherein removing the solvent method is accomplished by sublimation of the solvent at reduced pressure.

13. The method of claim 2, further comprising heating the resultant organic nanopowder product to about room temperature under vacuum conditions, and then bringing it to atmospheric pressure under a dry inert gas.

14. A method for producing ascorbic acid nanopowders comprising:
preparing a solution of ascorbic acid using an appropriate solvent;
rapidly freezing the ascorbic acid solution so that little or no phase separation occurs to form a frozen material comprising frozen solvent and immobilized ascorbic acid;
removing the solvent component of the frozen material from the frozen material while the frozen material is held at a temperature of from about equal to or less than the eutectic point of the solvent and ascorbic acid binary system;
recovering the ascorbic acid product after the solvent component is removed.

15. The method of claim 14, wherein the ascorbic acid produced has a particle size less than 100 nanometers.

16. The method of claim 14, wherein the ascorbic acid produced has a particle size less than 50 nanometers.

17. The method of claim 14, wherein the ascorbic acid produced has a particle size less than 25 nanometers.

18. The method of claim 14, wherein the ascorbic acid produced has a particle size less than 15 nanometers.

19. The method of claim 14, wherein the ascorbic acid produced has a particle size less than 10 nanometers.

20. A method for producing calcium ascorbate nanopowders comprising:
preparing a solution of calcium ascorbate using an appropriate solvent;
rapidly freezing the calcium ascorbate solution so that little or no phase separation occurs to form a frozen material comprising frozen solvent and immobilized calcium ascorbate;
removing the solvent component of the frozen material from the frozen material while the frozen material is held at a temperature of from about equal to or less than the eutectic point of the solvent and calcium ascorbate binary system;
recovering the calcium ascorbate product after the solvent component is removed.

21. The method of claim 20, wherein the appropriate solvent is water.

22. The method of claim 20, wherein rapidly freezing the calcium ascorbate solution is accomplished by dispersing the calcium ascorbate solution into aerosol droplets and rapidly mixing said solution with a cryogenic liquid, whereby the cryogenic liquid causes the aerosol droplets to freeze.

23. The method of claim 20, wherein the solvent removal is accomplished by sublimation.

24. The method of claim 20, wherein the solvent removal method is accomplished by sublimation at reduced pressure.

25. The method of claim 14, wherein the solvent and ascorbic acid binary system is a water ascorbic acid binary system.

26. The method of claim 20, further comprising heating the recovered ascorbic acid to about room temperature under vacuum conditions, and then bringing it to atmospheric pressure under a dry inert gas.

27. The method of claim 20, wherein the calcium ascorbate recovered has a particle size less than 100 nanometers.

28. The method of claim 20, wherein the calcium ascorbate recovered has a particle size less than 50 nanometers.

29. The method of claim 20, wherein the calcium ascorbate recovered has a particle size less than 25 nanometers.

30. The method of claim 20, wherein the calcium ascorbate recovered has a particle size less than 15 nanometers.

31. The method of claim 20, wherein the calcium ascorbate recovered has a particle size less than 10 nanometers.

32. The method of claim 13, wherein the ascorbic acid solution contains at least one ascorbic acid compound selected from the group consisting of: salts of ascorbic acid.

33. The method of claim 13, wherein the ascorbic acid solution contains at least one ascorbic acid compound selected from the group consisting of: sodium ascorbate, potassium ascorbate, and calcium ascorbate.

34. Ascorbic acid nanoparticle compounds produced according to the method of claim 13, wherein said ascorbic acid nanoparticle compounds have a particle size of less than about 100 nanometers.

35. The ascorbic acid nanoparticle compounds of claim 33 having a particle size of less than about 15 nanometers.

36. The method of claim 1, wherein the solvent and solute binary system is a water ascorbic acid binary system.

37. An organic nanopowder produced according to the method of claim 1 having a particle size less than 100 nanometers.

38. An organic nanopowder produced according to the method of claim 1 having a particle size less than 50 nanometers.

39. An organic nanopowder produced according to the method of claim 1 having a particle size less than 25 nanometers.

40. An organic nanopowder produced according to the method of claim 1 having a particle size less than 15 nanometers.

41. An organic nanopowder produced according to the method of claim 1 having a particle size less than 10 nanometers.

42. An organic nanopowder according to claim 37, wherein the organic nanopowder produced is a compound selected from the group consisting of ascorbic acid and salts thereof.

43. An organic nanopowder according to claim 42, wherein the organic nanopowder compound produced is calcium ascorbate.

44. The method of claim 42, wherein the ascorbic acid salts are used to produce nanoparticles comprised of salts of ascorbic acid.

45. An organic nanopowder according to claim 38, wherein the organic nanopowder produced is a compound selected from the group consisting of ascorbic acid and salts thereof.

46. An organic nanopowder according to claim 39, wherein the organic nanopowder produced is a compound selected from the group consisting of ascorbic acid and salts thereof.

47. An organic nanopowder according to claim 40, wherein the organic nanopowder produced is a compound selected from the group consisting of ascorbic acid and salts thereof.

48. An organic nanopowder according to claim 41, wherein the organic nanopowder produced is a compound selected from the group consisting of ascorbic acid and salts thereof.
